(19) United States
(12) Patent Application Publication
(10) Pub. No.: US 2025/0032376 A1
(43) Pub. Date: Jan. 30, 2025

(54) COSMETIC COMPOSITION COMPRISING HINOKITIOL

(71) Applicant: Conopco, Inc., d/b/a UNILEVER, Englewood Cliffs, NJ (US)

(72) Inventors: James MERRINGTON, West Kirby, Wirral (GB); Xuezhi TANG, Shanghai (CN); Ian Geoffrey WOOD, Chester (GB)

(73) Assignee: Conopco, Inc., d/b/a UNILEVER, Englewood Cliffs, NJ (US)

(21) Appl. No.: 18/716,671

(22) PCT Filed: Nov. 9, 2022
(86) PCT No.: PCT/EP2022/081223

§ 371 Date: Jun. 5, 2024

(30) Foreign Application Priority Data
Dec. 14, 2021 (WO) PCT/CN2021/137864
Jan. 20, 2022 (EP) 22152359.0

(51) Int. Cl. A61K 8/35; A61K 8/19; A61K 8/73; A61K 8/81; A61Q 5/00

(52) U.S. Cl. CPC A61K 8/35; A61K 8/19; A61K 8/73; A61K 8/8158; A61Q 5/00

(57) ABSTRACT

A cosmetic composition is disclosed comprising i) hinokitiol; ii) a metal oxide or metal salt wherein the metal is selected from manganese, stannum and barium; and iii) a cationic polymer selected from cationic galactomannans, dialkyldiallylammonium halide homopolymers and copolymers, cationic cellulose, (meth)acrylamidopropyl trialkylammonium halide homopolymers and copolymers, trialkylammonioethyl (meth)acrylate homopolymers and copolymers; wherein the weight ratio of the metal oxide or metal salt to hinokitiol is from 1:10 to 20:1.

### FIELD OF THE INVENTION

This invention relates to cosmetic compositions, preferably rinse off compositions, more preferably hair care compositions. More particularly the invention relates to cosmetic compositions that comprise hinokitiol.

### BACKGROUND OF THE INVENTION

Hinokitiol is a tropolone originally isolated from the heart wood of T. plicata that has diverse biological activities, including antifungal, antibacterial, and antiproliferative properties. Hinokitiol has been used in oral, hair and skin care products. Generally, hinokitiol is dissolved in the base composition of a product which includes surfactants, water, polymers and colouring and fragrance ingredients. When a user applies the compositions to hair, oral or skin, compound of hinokitiol get deposited thereon.

A common problem with such active is that deposition of the active onto the desired surface during wash process is minimum. The desired surface is typically skin, scalp and/or hair. During the excessive rinsing process, the majority of hinokitiol is likely to be washed away together with the surfactants. Consequently, there remains a need to improve the deposition of hinokitiol onto the surface of skin, scalp and/or hair during washing process.

The present inventors have now found unexpectedly that the combination of a specific metal oxide or metal salt and cationic polymer, can enhance deposition of hinokitiol onto the surface of skin, scalp and/or hair.

### SUMMARY OF THE INVENTION

In a first aspect, the present invention is directed to a cosmetic composition comprising:
i) a hinokitiol;
ii) a metal oxide or metal salt wherein the metal is selected from manganese, stannum and barium; and
iii) a cationic polymer selected from cationic galactomannans, dialkyldiallylammonium halide homopolymers and copolymers, cationic cellulose, (meth)acrylamidopropyl trialkylammonium halide homopolymers and copolymers, trialkylammonioethyl (meth)acrylate homopolymers and copolymers; wherein the weight ratio of the metal oxide or metal salt to hinokitiol is from 1:10 to 20:1.

In a second aspect, present invention is directed to a method of depositing hinokitiol onto skin, scalp and/or hair comprising the step of applying the cosmetic composition of the first aspect onto skin, scalp and/or hair surfaces of an individual followed by rinsing the surfaces with water.

In a third aspect, the present invention is directed to a use of the composition of the first aspect for enhancing deposition of hinokitiol onto skin, scalp and/or hair.

All other aspects of the present invention will more readily become apparent upon considering the detailed description and examples which follow.

### DETAILED DESCRIPTION OF THE INVENTION

### Cosmetic Composition

By ‘a cosmetic composition’ as used herein, is meant to include a composition for topical application to skin, hair and/or scalp of mammals, especially human beings. Such a composition is generally applied on to the desired topical surface of the body for a period of time from a few seconds to up to 24 hours. When the period of time of application is low say of the order of a few seconds to a few minutes after which the composition is rinsed off with water or wiped away, such a composition is known as a cleansing composition or a wash-off composition. On the other hand, When the composition is applied for longer period of time say from several minutes to up to 24 hours and washed off usually during the process of normal personal cleaning, such a composition is known as a leave-on composition. The composition as per the present invention includes any product applied to a human body for also improving appearance, cleansing, odor control or general aesthetics.

### Hair Care Composition

“Hair care composition”, as used herein, is meant to include a composition for topical application to hair and/or scalp of mammals, especially humans. Such a composition may be generally classified as leave-on or rinse off, and includes any product applied to a human body for also improving appearance, cleansing, odor control or general aesthetics. The composition of the present invention can be in the form of a liquid, lotion, cream, foam, scrub, gel, or bar. Non-limiting examples of such compositions include leave-on hair lotions, creams, and rinse-off shampoos, conditioners, shower gels, or toilet bar. The composition of the present invention is preferably a rinse-off composition, especially preferred being a shampoo or a conditioner and most preferably a shampoo.

### Cationic Charge Density

“Cationic charge density”, as used herein, refers to the number of cationic charges per weight unit of a given polymer. Cationic charge density can be calculated from the degree of substitution as described in WO 2013/011122, the disclosure of which is hereby incorporated by reference in its entirety but especially page 8 lines 8-17. For example, for cationic guar polymer obtained by reacting with 2,3-epoxypropyltrimethylammonium chloride, the cationic charge density may be calculated from the degree of substitution using the following equation:

$$Cationic charge density in milliequivalents per gram ( meq / g ) = \frac{D S \times 1000}{162 + 151 \times D S}$$

### Molecular Weight

“Molecular weight”, as used herein, refers to the weight average molecular mass of a given polymer. The weight average molecular weight (WAVG MW) of a given polymer is determined by SEC (Size Exclusion Chromatography) analysis using absolute calibration (universal calibration). Polysaccharide standards pulluan and dextran were used for calibration.

### Miscellaneous

Except in the examples, or where otherwise explicitly indicated, all numbers in this description indicating amounts of material or conditions of reaction, physical properties of materials and/or use may optionally be understood as modified by the word “about”.

All amounts are by weight of the final hair care composition, unless otherwise specified. It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### Hinokitiol

Hinokitiol (beta-thujaplicin) according to the present invention is a monoterpenoid that is cyclohepta-2,4,6-trien-1-one substituted by a hydroxy group at position 2 and an isopropyl group at position 4. The CAS number is 499-44-5 and the compound has the general formula (1) as below:

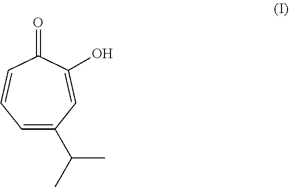

Amount of the hinokitiol in the composition of the invention would depend on the type of the cosmetic composition and the precise nature of other antimicrobial agents used. It is preferred that the composition comprises from 0.01 to 10% of said hinokitiol, preferably from 0.05 to 5%, more preferably from 0.1 to 3%, furthermore preferably from 0.2 to 2% by weight of the composition.

### Cationic Polymer

The composition of the present invention comprises a cationic polymer selected from cationic galactomannans, cationic cellulose, dialkyldiallylammonium halide homopolymers and copolymers, (meth)acrylamidopropyl trialkylammonium halide homopolymers and copolymers, trialkylammonioethyl (meth)acrylate homopolymers and copolymers.

It is preferred that the composition comprises cationic polymer having a molecular weight of from 50 k-3000 k Dalton and a charge density of 0.5-7 meg/g.

It is preferred that the cationic polymer has a molecular weight of from 1.0 to 2.5 million Dalton, more preferably from 1.5 to 2.2 million Dalton.

When the cationic polymer is a cationic galactomannan gum, it can be selected from the group consisting of cationic guar, tara, cassia, locust bean and fenugreek gums, preferably the cationic galactomannan gum is fenugreek gums.

Alternatively, the cationic polymer can be a cationic cellulose. Cationic cellulose is available, for example, from the Dow Chemical Company in their UCARE JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from the Dow Chemical Company under the tradename Polymer LM-200. Most preferred is Polyquaternium 10, especially Polyquaternium 10 with a molecular weight (weight average by GPC) in the range 0.5 to 3 million Daltons and charge density in the range 0.5 to 3 meq/g. The preferred Polyquaternium 10 is available, for example, as UCARE™ JR30M from Dow Chemical.

The cationic polymer can be one of dialkyldiallylammonium halide homopolymers. It comprises the repeating units (II) as below:

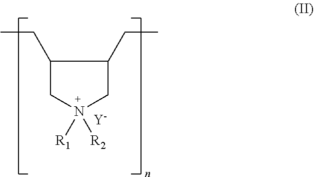

R1, R2: methyl or other alkyls; Y: Cl, Br or I

Preferably, it can be one of Polyquaternium-6 (PDADMAC), Polyquaternium-7, Polyquaternium-4, Polyquaternium-22 and Polyquaternium-39. More preferably it is Polyquaternium-6 (PDADMAC).

The cationic polymer can be one of (meth)acrylamidopropyl trialkylammonium halide homopolymers and copolymers. It comprises the repeating units (Ill) as below:

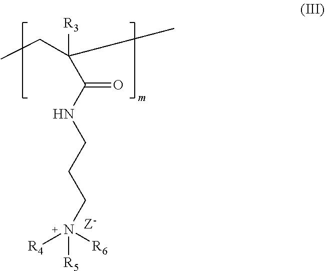

R3: H or methyl
R4, R5, R6: methyl or other alkyls
Z: Cl, Br or I

Preferably, it can be one of polyacrylamidopropyltrimonium chloride, acrylamidopropyltrimonium chloride/acrylamide copolymer, polyquaternium-28, polyquaternium-43, polyquaternium-47, polyquaternium-53, polyquaternium-55, polyquaternium-69, polyquaternium-74, polyquaternium-113. More preferably, it is acrylamidopropyltrimonium chloride/acrylamide copolymer. An example of a suitable copolymer is commercially available from Ashland under the trade name N-Hance SP-100@. N-Hance SP-100@ has a charge density of from 1.8 to 2.2 meq/g and a molecular weight of from 1,800,000 to 2,200,000 g/mol.

The cationic polymer can be one of trialkylammonioethyl (meth)acrylate homopolymers and copolymers. It comprises the repeating units (IV) as below:

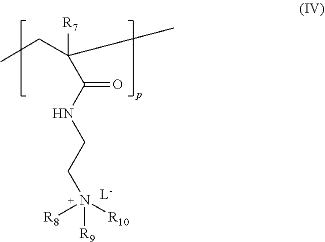

R7: H or methyl
R8, R9, R10: methyl or other alkyls
L: Cl, Br, I or CH3SO3

Preferably, it can be one of Polyquaternium-5, Polyquaternium-11, Polyquaternium-37.

Generally for cationic polysaccharide polymers, the hydroxyl groups of the non-modified monomeric sugar ring units are the sites for cationic substitution. Degree of substitution (DS) is typically in the range from 0 to 3 due to the fact that the monomeric sugar unit of most polysaccharide has in average three hydroxyl groups available for substitution. In addition to the DS, the cationic charge on polymers can also be quantified as cationic charge density. DS has previously been determined by different methods. For example, the cationic charge density of the polymer has in some cases been calculated based on a percent nitrogen content determined via the Kjeldahl method as described in US Pharmacopoeia under chemical tests for nitrogen determination and is expressed in milliequivalents (meq) per gram. The cationic charge density of the polymer in the present invention is, however, calculated from the degree of substitution, which is measured by 1H NMR in a solvent of deuterium oxide (D20) and deuterium chloride (DCI) mixture.

In many cases the DS obtained from 1H NMR measurement may not be suitable to be compared with that obtained from Kjeldahl method, due to the fact that the two methods are influenced by different factors.

In the wide spectrum of molecular weights of guars available, the cationic guar for use in the present invention has a molecule weight which is considered a ‘medium’ molecular weight. In the wide range of charge densities, the above range for use in the present invention is considered a ‘medium’ range.

The cationic polymer is preferably present in an amount of from 0.01 to 5%, more preferably from 0.05 to 2%, furthermore preferably 0.1 to 2%, most preferably from 0.2 to 1% by weight of the composition.

### Metal Oxide or Metal Salt

The composition of the present invention comprises a metal oxide or metal salt wherein the metal is selected from manganese, stannum and barium.

When the composition comprises a metal oxide, it is preferably selected form manganese oxide, stannum oxide and barium oxide.

The metal salt is suitable for cosmetic use. A metal salt for instance is selected from metal carboxylates, metal halids, metal sulphadiazine, metal sulphate, metal nitrate, metal carbonate, metal carbonate hydroxide, metal oxychloride, metal hydroxide, metal acetylacetonate, metal PCA (pyrrolidone carboxylic acid), metal PCA methylsilanol, metal acetyl tyrosinate methylsilanol, metal acetylmethionate, metal aminoacetylamidoimidazolyl propionate, metal picolinate, metal peptides, metal ATP, metal DNA, metal amino acid salts, metal silicates, metal quinolines and derivatives; metal pyrithione and other metal salts of pyridine thiols; metal phosphates, chlorophyllin metal complexes; metal chlorophyll; disodium EDTA metal; and mixtures thereof.

Suitable metal carboxylates include lactate, citrate, ascorbate, acetate, gluconate, laurate, myristate, palmitate, salicylate, aspirinate, stearate, succinate, tartrate, undecylenate, usnate, neodecanoate and ricinoleate.

Suitable metal halides include metal chloride, metal bromide and metal iodide.

More preferably, the composition of the present invention comprises a water-soluble metal salt, preferably selected from manganese chloride, manganese bromide, manganese iodide, manganese nitrate, stannum chloride, stannum bromide, stannum iodide, stannum nitrate, barium chloride, barium bromide, barium iodide and barium nitrate It is preferred that the metal oxide or metal salt of the present invention is present in an amount of from 0.1 to 10%, more preferably from 0.2 to 5%, furthermore preferably from 0.5 to 2% by weight of the composition.

Without wishing to be bound by theory the inventors believe that the hinokitiol can form complex with these specific metal oxide or metal salt, therefore, the deposition of hinokitiol can be improved with the presence of specific cationic polymer.

The weight ratio of metal oxide or metal salt to hinokitiol in the composition is from 1:10 to 20:1, preferably from 1:10 to 10:1, more preferably from 1:5 to 5:1.

### Cosmetic Composition

The composition of the invention preferably comprises a cosmetically acceptable carrier. The cosmetically acceptable carrier is such that the composition can be prepared, e.g., as a shampoo, conditioner, body wash, hand wash or face wash product, cream, lotion, gel, powder, ointment, deodorant, hand sanitiser or a soap bar and the rest of the ingredients would vary accordingly.

It is preferred that the composition is a skin or hair product including rinse-off and leave-on product, more preferably a hair care composition. More preferably the composition is a rinse-off product. It is preferred that the composition comprises a cleansing surfactant.

The composition of this invention preferably comprises a cleansing surfactant comprising an anionic surfactant.

Non-limiting examples of suitable anionic surfactants are alkyl sulphates, alkyl ether sulphates, alkaryl sulphonates, alkanoyl isethionates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulphates, alkyl ether sulphosuccinates, alkyl ether phosphates and alkyl ether carboxylic acids and salts thereof may contain from 1 to 20 ethylene oxide or propylene oxide units per molecule. Typical anionic surfactants for use in compositions of the invention include, but not limited to, sodium oleyl succinate, ammonium lauryl sulphosuccinate, sodium lauryl sulphate, sodium lauryl ether sulphate, sodium lauryl ether sulphosuccinate, ammonium lauryl sulphate, sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate or mixtures thereof.

Preferred anionic surfactants are the alkyl sulphates and alkyl ether sulphates. Preferred alkyl sulphates are C8-18 alky sulphates, more preferably C12-18 alkyl sulphates, preferably in the form of a salt with a solubilising cation such as sodium, potassium, ammonium or substituted ammonium. Examples are sodium lauryl sulphate (SLS) or sodium dodecyl sulphate (SDS). It is particularly preferred that the anionic surfactant is alkyl ether sulphate. Preferred alkyl ether sulphates are those having the formula: RO(CH2CH2O)nSO¬3M; wherein R is an alkyl or alkenyl having from 8 to 18 (preferably 12 to 18) carbon atoms; n is a number having an average value of greater than at least 0.5, preferably between 1 and 3, more preferably between 1 and 2; and M is a solubilising cation such as sodium, potassium, ammonium or substituted ammonium. An example is sodium lauryl ether sulphate (SLES). Preferred alkyl ether sulphate is sodium lauryl ether sulphate having an average degree of ethoxylation of from 0.5 to 3, preferably from 1 to 3, more preferably from 1 to 2.

It is preferred that the anionic surfactant is an ethoxylated alkyl sulphate a degree of ethoxylation of less than 3, preferably an ethoxylated alkyl sulphate a degree of ethoxylation of less than 2, more preferably sodium laureth sulphate (1EO).

The anionic surfactants are typically present in the composition of the present invention at a level of from 0.5 to 20%, more preferably from 2 to 16% and most preferably from 3 to 16%, by weight of the composition.

Suitable amphoteric surfactant examples include alkyl amine oxides, alkyl betaines, alkyl amidopropyl betaines, alkyl sulphobetaines (sultaines), alkyl amphoacetates, alkyl amphopropionates, alkyl amidopropyl hydroxysultaines, wherein the alkyl group has from 8 to 19 carbon atoms.

Preferably, the amphoteric surfactant is a betaine surfactant. It is preferred that the betaine surfactant is one of alkyl amidopropyl betaines. Cocamidopropyl betaine (CAPB) is particularly preferred.

In a preferred embodiment, the composition comprises from 0.1 to 10 wt. %, preferably from 0.5 to 8 wt. %, more preferably from 1 to 5 wt. % of an amphoteric surfactant by weight of the composition.

The ratio of ethoxylated anionic surfactant to amphoteric surfactant in which the ratio of anionic surfactant to amphoteric surfactant is less than 10:1, preferably from 2:1 to 10:1, more preferably from 3:1 to 9:1.

The total amount of cleansing surfactant in the composition for use in the invention is generally from 3 to 35 wt %, preferably from 5 to 25 wt %, most preferably from 7 to 16 wt % of the total composition.

### Hair Care Composition

Preferably the composition is a shampoo, hair conditioner, hair cream, hair gel, hair serum, mousse or hair oil.

In a preferred embodiment, the hair care composition is a shampoo, particularly a rinse off shampoo. Shampoo compositions for use in the invention are generally aqueous, i.e. they have water or an aqueous solution or a lyotropic liquid crystalline phase as their major component. Suitably, the shampoo composition will comprise from 50 to 98%, preferably from 60 to 92% water by weight based on the total weight of the composition. Such compositions are referred to as having an aqueous base.

Antidandruff agents are compounds that are active against dandruff and are typically antimicrobial agents, preferably antifungal agents. Antidandruff agents typically display a minimum inhibitory concentration of about 50 mg/ml or less against Malassezia.

In addition to hinokitiol, the hair care composition in accordance with this invention may also comprise free additional antidandruff agent which is different from it. Whenever present, the hair care composition of the invention preferably comprises 0.1 to 5 wt %, more preferably 0.5 to 2% of the additional antidandruff agent.

The additional antidandruff agent is preferably selected from piroctone compound, azoles, selenium sulfide, salicylic acid and combinations thereof. Azoles include ketoconazole and climbazole, preferably climbazole. It is preferred that the additional antidandruff agent is piroctone compound, more preferably piroctone olamine.

The hair care composition may additionally comprise a conditioning agent to provide conditioning benefit. Preferably, the hair care composition comprises discrete dispersed droplets of a water-insoluble conditioning agent, which has a mean droplet diameter (D3,2) of less than 15 microns, preferably less than 10 microns, more preferably less than 5 microns, most preferably less than 3 microns. The mean droplet diameter (D3,2) of a water-insoluble conditioning agent may be measured by means of a laser light scattering technique, for example using a 2600D Particle Sizer from Malvern Instruments.

The water-insoluble conditioning agent may include non-silicone conditioning agent comprising non-silicone oily or fatty materials such as hydrocarbon oils, fatty esters and mixtures thereof. Preferably, the water-insoluble conditioning agent is emulsified silicone oil.

Suitable silicones include polydiorganosiloxanes, in particular polydimethylsiloxanes which have the CTFA designation dimethicone. Also suitable for use in compositions of this invention (particularly shampoos and conditioners) are polydimethyl siloxanes having hydroxyl end groups, which have the CTFA designation dimethiconol. Also suitable for use in compositions of this invention are silicone gums having a slight degree of cross-linking, as are described for example in WO 96/31188. Preferably, the silicone oil comprises dimethicone, dimethiconol or a mixture thereof.

Suitable emulsified silicones for use in the hair care compositions of this invention are available as pre-formed silicone emulsions from suppliers of silicones such as Dow Corning and GE silicones. The use of such pre-formed silicone emulsion is preferred for ease of processing and control of silicone particle size. Such pre-formed silicone emulsions will typically additionally comprise a suitable emulsifier, and may be prepared by a chemical emulsification process such as emulsion polymerisation, or by mechanical emulsification using a high shear mixer. Examples of suitable pre-formed silicone emulsions include DC1785, DC1788, DC7128, all available from Dow Corning. These are emulsions of dimethiconol/dimethicone.

Another class of silicones which may be used are functionalized silicones such as amino functional silicones, meaning a silicone containing at least one primary, secondary or tertiary amine group, or a quaternary ammonium group. Examples of suitable amino functional silicones include polysiloxanes having the CTFA designation “amodimethicone.”

The water-insoluble conditioning agent is generally present in hair care composition of this invention in an amount from 0.05 to 15%, preferably from 0.1 to 10%, more preferably from 0.5 to 8%, most preferably from 1 to 5%, based on total weight of the hair care composition.

Preferably the composition of the invention further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

Mixtures of any of the above suspending agents may be used. Preferred is a mixture of cross-linked polymer of acrylic acid and crystalline long chain acyl derivative.

The suspending agent is generally present in hair care composition of this invention in an amount of from 0.1 to 10%, more preferably from 0.2 to 6%, and most preferably from 0.3 to 4%, based on total weight of the hair care composition and including all ranges subsumed therein.

Preservatives may also be incorporated into the hair care composition of this invention to protect against the growth of potentially harmful microorganisms. Suitable traditional preservatives include alkyl esters of parahydroxybenzoic acid, hydantoin derivatives, propionate salts, and a variety of quaternary ammonium compounds. Illustrative yet non-limiting examples of the types of preservatives that may be used in this invention include, for examples, phenoxyethanol, sodium salicylate, methyl paraben, butyl paraben, propyl paraben, diazolidinyl urea, sodium dehydroacetate, benzyl alcohol, sodium benzoate, iodopropynyl butylcarbamate, caprylyl glycol, disodium EDTA or mixtures thereof. In an especially preferred embodiment, the preservative is sodium benzoate, phenoxyethanol, sodium salicylate or a mixture thereof. Preservatives are preferably employed in amounts ranging from 0.01 to 2% by weight of the hair care composition.

It is further preferred that the hair care composition of the invention comprises a cosmetic ingredient. Preferably the cosmetic ingredient is selected from the group consisting of a silicone, an antibacterial agent other than antidandruff agents, a foam booster, a perfume, encapsulates (for example encapsulated fragrance) a dye, a colouring agent, a pigment, a preservative, a thickener, a protein, a phosphate ester, a buffering agent, a pH adjusting agent, a pearlescer (for example; mica, titanium dioxide, titanium dioxide coated mica, ethylene glycol distearate (INCI glycol distearate)) and/or opacifier, a viscosity modifier, an emollient, a sunscreen, an emulsifier, a sensate active (for example menthol and menthol derivatives), vitamins, mineral oils, essential oils, lipids, natural actives, glycerin, natural hair nutrients such as botanical extracts, fruit extracts, sugar derivatives and amino acids, microcrystalline cellulose and mixtures thereof.

Preferably, the composition of the present invention includes from 0.01 to 20 wt % of the at least one cosmetic ingredient, more preferably from 0.05 to 10 wt %, still more preferably from 0.075 to 7.5 wt % and most preferably, from 0.1 to 5 wt % of the at least one cosmetic ingredient, by weight of the total composition.

The composition of the present invention may also comprise synergistic antimicrobial compounds that give synergistic antimicrobial benefit when used in combination with the antimicrobial active to enhance its properties and further inhibit the growth of bacterial/fungal. Non-limiting examples of these compounds include compounds having alcoholic groups (e.g. honokiol, magnolol or paeonol), Piperazines and a phenolic compound found in natural plant extract viz. thymol and terpeniol.

### Method and Use

The present invention provides for a method of depositing hinokitiol onto skin, scalp and/or hair comprising the step of applying the composition of the first aspect onto skin, scalp and/or hair surfaces of an individual followed by rinsing the surfaces with water. The method is non-therapeutic in nature. Preferably it is for cosmetic purpose.

The present invention also provides for use of the composition of the first aspect for enhancing deposition of hinokitiol onto skin, scalp and/or hair. The use is non-therapeutic in nature. Preferably it is for cosmetic purpose.

The present invention also provides for a composition of the first aspect for use in preventing or alleviating the symptoms of dandruff on the scalp or hair.

The present invention also provides a composition of the first aspect for use in a method to mitigate dandruff.

### Mode of Use

The compositions of the invention are primarily intended for topical application to scalp and/or at least a portion of the hair of an individual, either in rinse-off or leave-on compositions, preferably in rinse-off compositions like shampoos.

When the composition is a shampoo, it is topically applied to the hair and then massaged into the hair and scalp. Then it is rinsed with water prior to drying the hair.

The invention will be further illustrated by the following, non-limiting Examples, in which all percentages quoted are by weight based on total weight unless otherwise stated.

The examples are intended to illustrate the invention and are not intended to limit the invention to those examples per se.

### EXAMPLES

### Example 1

This example demonstrated the combination of stannous salt and cationic polymer within the scope of the present invention can improve the deposition of hinokitiol. The compositions according to the formulations detailed in Table 1 are prepared. All ingredients are expressed by weight percent of the total formulation, and as level of active ingredient.

TABLE 1

| | Samples | | | | | |
|---|---|---|---|---|---|---|
| Ingredient | A | B | C | D | 1 | 2 |
| Water | To 100 | To 100 | To 100 | To 100 | To 100 | To 100 |
| Sodium lauryl sulphate | 13.00 | 13.00 | 13.00 | 13.00 | 13.00 | 13.00 |
| Cocoamidopropyl betaine | 1.00 | 1.00 | 1.00 | 1.00 | 1.00 | 1.00 |
| Hinikitiol | 1.00 | 1.00 | 1.00 | 1.00 | 1.00 | 1.00 |
| Stannous chloride dihydrate | — | 1.03 | — | — | 1.03 | 1.03 |
| Poly(dimethyl diallyl | — | — | 0.2 | — | 0.2 | — |
| ammonium chloride)^{a} | | | | | | |
| Acryl amidopropyltrimonium | — | — | — | 0.2 | — | 0.2 |
| Chloride/Acrylamide Copolymer^{b} | | | | | | |
| Preservative | 0.80 | 0.80 | 0.80 | 0.80 | 0.80 | 0.80 |
| PH adjuster | 1.25 | 1.25 | 1.25 | 1.25 | 1.25 | 1.25 |

^{a}Merquat ™ 100 Polymer from Lubrizol.
^{b}N-DurHance ™ SP-100 Polymer from Ashland.

### Methods

A typical deposition evaluating protocol is used as follows.

### Switch Wash:

(1) Rinse the hair switches (2.5 g/6″ Double Bleached European) in their sets of 3 for 30 seconds under tap (flow rate 4 L/min and room temperature).
(2) Lay the set of 3 switches down flat on a surface next to sink and apply of required shampoo, 0.75 g, (0.1 g/g of hair for 3 switches) along switches using a 1 mL syringe. Ensure even coverage of shampoo (should be around 0.25 g per hair switch).
(3) Massage shampoo into switches for 30 seconds.
(4) Rinse switches for 30 seconds running fingers through every 5 seconds
(5) Repeat steps (2)-(4)
(6) After second rinse do not comb. Leave to dry at 50° C. for 90 minutes ensuring protection of hair switches from light such as covering oven doors with aluminum foil.
(7) Repeat the process for the remaining sets of 3 switches (each set of 3 switches will be washed by one shampoo).

### Switch Extraction:

(1) Cut each dry hair switch (excluding metal clip) into 60 ml amber jars. This can be done by wrapping switch around gloved finger.
(2) Add 20 mL extraction solvent (in this case methanol) to jar, seal and place for 1 hour.
(3) After 1 hour, remove 1 mL extracting methanol solution and pass through a 0.2 μm PTFE filter to an amber UPLC screw cap vial.
(4) Analyse using appropriate UPLC method.

$$Hinokitiol deposition efficacy = ( amount of deposited hinokitiol measured by UPLC ) / \\ ( theoretical total amount of hinokitiol applied on hair ) * 100 \%$$

### Results

The average deposition (of three such experiments) is summarized in Table 2 (error represents standard deviation for duplicate measurements).

TABLE 2

| Samples | A | B | C | D | 1 | 2 |
|---|---|---|---|---|---|---|
| Hinokitiol | 1.40 ± | 0.91 ± | 1.47 ± | 1.81 ± | 2.72 ± | 2.68 ± |
| deposition | 0.43 | 0.12 | 0.50 | 0.57 | 0.42 | 0.21 |
| efficacy (%) | | | | | | |

Samples 1 and 2 comprising combination of stannous salt and cationic polymer within the scope of the present invention showed improved deposition of hinokitiol as compared to samples A-D which are outside the scope of the present invention.

### Example 2

This example demonstrated the combination of manganese salt and cationic polymer within the scope of the present invention can improve the deposition of hinokitiol. The compositions according to the formulations detailed in Table 3 are prepared. All ingredients are expressed by weight percent of the total formulation, and as level of active ingredient.

TABLE 3

| | Samples | | | | | | | |
|---|---|---|---|---|---|---|---|---|
| Ingredient | E | F | G | H | I | 3 | 4 | 5 |
| Water | To 100 | To 100 | To 100 | To 100 | To 100 | To 100 | To 100 | To 100 |
| Sodium lauryl sulphate | 13.00 | 13.00 | 13.00 | 13.00 | 13.00 | 13.00 | 13.00 | 13.00 |
| Cocoamidopropyl betaine | 1.00 | 1.00 | 1.00 | 1.00 | 1.00 | 1.00 | 1.00 | 1.00 |
| Hinikitiol | 1.00 | 1.00 | 1.00 | 1.00 | 1.00 | 1.00 | 1.00 | 1.00 |
| Manganese chloride | — | 0.58 | — | — | — | 0.58 | 0.58 | 0.58 |
| Acryl amidopropyltrimonium | — | — | 0.2 | — | — | 0.2 | — | — |
| Chloride/Acrylamide Copolymer^{b} | | | | | | | | |
| Cationic fenugreek gum^{c} | — | — | — | 0.2 | — | — | 0.2 | — |
| Polyquaternium-10^{d} | — | — | — | — | 0.2 | — | — | 0.2 |
| Preservative | 0.80 | 0.80 | 0.80 | 0.80 | 0.80 | 0.80 | 0.80 | 0.80 |
| PH adjuster | 1.25 | 1.25 | 1.25 | 1.25 | 1.25 | 1.25 | 1.25 | 1.25 |

^{b}N-DurHance ™ SP-100 Polymer from Ashland.
^{c}Trade name Catinal CF-100 from TOHO Chemical Japan.
^{d}Trade name UCARE ™ Extreme Polymer from Dow Chemical.

### Methods

Same deposition evaluating method was used as disclosed in Example 1.

### Results

The average deposition (of three such experiments) is summarized in Table 4 (error represents standard deviation for duplicate measurements).

TABLE 4

| Samples | E | F | G | H | I | 3 | 4 | 5 |
|---|---|---|---|---|---|---|---|---|
| Hinokitiol | 1.40 ± | 0.79 ± | 1.81 ± | 1.50 ± | 1.52 ± | 20.90 ± | 4.32 ± | 8.00 ± |
| deposition | 0.43 | 0.40 | 0.57 | 0.32 | 0.39 | 9.65 | 0.81 | 3.79 |
| efficacy (%) | | | | | | | | |

Samples 3-5 comprising combination of manganese salt and cationic polymer within the scope of the present invention showed improved deposition of hinokitiol as compared to samples E-1 which are outside the scope of the present invention.

### Example 3

This example demonstrated the combination of manganese salt and cationic polymer outside the scope of the present invention cannot improve the deposition of hinokitiol. The compositions according to the formulations detailed in Table 5 are prepared. All ingredients are expressed by weight percent of the total formulation, and as level of active ingredient.

TABLE 5

| | Samples | | | | | |
|---|---|---|---|---|---|---|
| Ingredient | E | F | J | K | L | M |
| Water | To 100 | To 100 | To 100 | To 100 | To 100 | To 100 |
| Sodium lauryl sulphate | 13.00 | 13.00 | 13.00 | 13.00 | 13.00 | 13.00 |
| Cocoamidopropyl betaine | 1.00 | 1.00 | 1.00 | 1.00 | 1.00 | 1.00 |
| Hinikitiol | 1.00 | 1.00 | 1.00 | 1.00 | 1.00 | 1.00 |
| Manganese chloride | — | 0.58 | — | — | 0.58 | 0.58 |
| Polylysine^{e} | — | — | 0.2 | — | 0.2 | — |
| Hydroxypropyltrimonium | — | — | — | 0.2 | — | 0.2 |
| Inulin^{f} | | | | | | |
| Preservative | 0.80 | 0.80 | 0.80 | 0.80 | 0.80 | 0.80 |
| PH adjuster | 1.25 | 1.25 | 1.25 | 1.25 | 1.25 | 1.25 |

^{e}Trade name Everguard ™ PL from Sino Lion
^{f}Trade name QUATIN ® 680 TQ-D from Cosun Beet Company

### Methods

Same deposition evaluating method was used as disclosed in Example 1.

### Results

The average deposition (of three such experiments) is summarized in Table 6 (error represents standard deviation for duplicate measurements).

TABLE 6

| Samples | E | F | J | K | L | M |
|---|---|---|---|---|---|---|
| Hinokitiol | 1.40 ± | 0.79 ± | 1.31 ± | 1.18 ± | 0.96 ± | 0.90 ± |
| deposition | 0.43 | 0.40 | 0.25 | 0.28 | 0.40 | 0.40 |
| efficacy (%) | | | | | | |

The results of samples L and M above indicates that compositions comprising manganese salt and cationic polymer outside the scope of the present invention cannot improve the deposition of the hinokitiol.

1. A cosmetic composition comprising:
i) hinokitiol;
ii) a metal oxide or a metal salt wherein the metal is selected from manganese, stannum and barium; and
iii) a cationic polymer selected from cationic galactomannan gums,
dialkyldiallylammonium halide homopolymers and copolymers, cationic cellulose, (meth)acrylamidopropyl trialkylammonium halide homopolymers and copolymers, trialkylammonioethyl (meth)acrylate homopolymers and copolymers,
wherein a weight ratio of the metal oxide or the metal salt to hinokitiol is from 1:10 to 20:1.

2. The composition as claimed in claim 1, wherein the weight ratio of the metal salt to hinokitiol is from 1:10 to 10:1.

3. The composition as claimed in claim 1, wherein the cationic polymer is dialkyldiallylammonium halide homopolymers and copolymers.

4. The composition as claimed in claim 1, wherein the cationic polymer is (meth)acrylamidopropyl trialkylammonium halide homopolymers and copolymers.

5. The composition as claimed in claim 1, wherein the cationic polymer is cationic galactomannans.

6. The composition as claimed in claim 1, wherein the cationic polymer is a cationic cellulose.

7. The composition as claimed in claim 1, wherein the metal salt is water soluble.

8. The composition as claimed in claim 1, wherein the composition comprises hinokitiol in an amount of from 0.01 to 10%.

9. The composition as claimed in claim 1, wherein the composition comprises the cationic polymer in an amount of from 0.01 to 5% by weight of the composition.

10. The composition as claimed in claim 1, wherein the composition comprises the metal oxide or metal salt in an amount from 0.1 to 10% by weight of the composition.

11. The composition as claimed in claim 1, wherein the composition further comprises a cleansing surfactant.

12. The composition as claimed in claim 11, wherein said cleansing surfactant comprising an anionic surfactant.

13. The composition as claimed in claim 1, wherein said composition is a hair care composition.

14. A method of depositing hinokitiol onto skin, scalp and/or hair comprising: applying the composition as claimed in claim 1 onto skin, scalp and/or hair surfaces of an individual, and rinsing the skin, scalp and/or hair surfaces with water.

15. (canceled)

16. The composition as claimed in claim 2, wherein the weight ratio of the metal salt to hinokitiol is from 1:5 to 5:1.

17. The composition as claimed in claim 3, wherein the dialkyldiallylammonium halide homopolymers and copolymers is poly (dimethyl diallyl ammonium chloride).

18. The composition as claimed in claim 4, wherein the (meth)acrylamidopropyl trialkylammonium halide homopolymers and copolymers is an acryl amidopropyltrimonium chloride/acrylamide copolymer.

19. The composition as claimed in claim 5, wherein the cationic galactomannans, is a cationic fenugreek gum.

20. The composition as claimed in claim 6, wherein the cationic cellulose is Polyquaternium 10.

21. The composition as claimed in claim 7, wherein the metal salt is selected from manganese chloride, manganese bromide, manganese iodide, manganese nitrate, stannum chloride, stannum bromide, stannum iodide, stannum nitrate, barium chloride, barium bromide, barium iodide and barium nitrate.
